SEQUENCE LISTING (3 sequences)

SEQ ID NO: 1 - DNA, 22 bp, Homo sapiens
ugggucuuugcgggcgagauga

SEQ ID NO: 2 - DNA, 29 bp, Artificial Sequence
  Synthetic
cgaagcttctgcggtggggaggctttggt

SEQ ID NO: 3 - DNA, 29 bp, Artificial Sequence
  Synthetic
ccggatccggtcccgtctgtccactcaac

(19) United States
(12) Patent Application Publication
(10) Pub. No.: US 2022/0296629 A1
(43) Pub. Date: Sep. 22, 2022

(54) HUMAN HEAD AND NECK CANCER TREATMENT

(71) Applicant: CARMEL HAIFA UNIVERSITY ECONOMIC CORPORATION LTD., Haifa (IL)

(72) Inventors: Fuad FARES, Horfiesh (IL); Reem MIARI, Sakhnin (IL)

(21) Appl. No.: 17/616,107

(22) PCT Filed: Jun. 3, 2020
(86) PCT No.: PCT/IL2020/050619

§ 371 Date: Dec. 2, 2021

Related U.S. Application Data
(60) Provisional application No. 62/856,332, filed on Jun. 3, 2019.

(51) Int. Cl. A61K 31/7105; C12N 15/113; C12N 15/86; A61P 35/00

(52) U.S. Cl. CPC A61K 31/7105; C12N 15/113; C12N 15/86; A61P 35/00; C12N 2310/141; C12N 2320/30; C12N 2710/10343; C12N 2750/14143

(57) ABSTRACT

The present invention is directed to an expression vector and a composition comprising same, wherein the expression includes a precursor of miR-193 a. Further provided are methods of using the expression vector of the invention or the composition comprising same, such as in diagnosing and/or treating head and neck squamous cell carcinoma, in a subject in need thereof.

### CROSS-REFERENCE TO RELATED APPLICATIONS

This application claims the benefit of priority of U.S. Provisional Patent Application No. 62/856,332 titled “HUMAN HEAD AND NECK CANCER TREATMENT”, filed Jun. 3, 2019, the contents of which are incorporated herein by reference in their entirety.

### FIELD OF INVENTION

The present invention is in the field of cancer treatment using microRNA.

### BACKGROUND

Head and neck squamous cell carcinoma (HNSCC) constitutes a major public health burden with an annual incidence of almost 600,000 patients worldwide and a mean 5-year survival rate of less than 50%. Although the principle risk factors for HNSCC remain tobacco and alcohol use, human papilloma virus infection (HPV) has recently been found to be etiologically associated with 20-25% of HNSCC, mostly in the oropharynx.

The examination of epigenetic aberrations is a key factor in the diagnosis and treatment of malignancies, since they can serve as biomarkers for assessing cancer prognosis and response to treatment. Therefore, epigenetic modifications mainly promoter methylations are an emerging field for the use as biomarkers in early cancer detection, and unlike genetic mutations, these modifications are reversible, making them on the other hand attractive targets for therapeutic intervention.

Over the past decade, a class of small, non-coding, single-stranded RNAs known as microRNAs (miRNAs) have emerged as major regulators of the initiation and progression of human cancers. The up-regulation of oncogenic miRNAs (targeting tumor suppressor genes) and the down-regulation of tumor-suppressive miRNAs (targeting oncogenes) lead to the dysfunction of cancer cells, including malignant proliferation, invasion, and metastasis.

The expression pattern of mir-193a in several cancers including leukemia, hepatocarcinoma, lung epithelial carcinoma and cervical adenocarcinoma cell lines showed down-regulation of mir-193a resulting from epigenetic silencing. The expression of this miR in head and neck cancer cells was referred to in a study from 2008, in which miRNAs expression pattern in oral cancer was explored and results showed 54 micro-RNAs from a panel of 148 tested which were down regulated in 18 cell lines of OSCC. In the AML1/ETO-positive leukemia cells, epigenetic silencing of miR-193a expanded the oncogenic activity of the AML-ETO fusion protein.

### SUMMARY

The present invention provides expression vectors, compositions and methods comprising miR-193a for use in diagnosing and/or treating HNSCC disease.

According to a first aspect, there is provided a method of treating or ameliorating a subject afflicted with HNSCC, the method comprising the steps of: (a) determining the level of miR-193a in a sample obtained or derived from a subject, wherein downregulated expression of the miR-193a compared to a control is indicative of the subject is suitable for treatment using miR-193a, and, (b) administering a therapeutically effective amount of the miR-193a to the subject, thereby treating or ameliorating a subject afflicted with HNSCC.

According to another aspect, there is provided an expression vector comprising a promoter operably linked to a polynucleotide having a miR-193a precursor sequence, for use in the treatment of head and neck squamous cell carcinoma (HNSCC) in a subject in need thereof.

According to another aspect, there is provided a pharmaceutical composition comprising an effective amount of any one of: (a) miR-193a or a precursor thereof and (b) the expression vector of the invention, and a pharmaceutically acceptable carrier, for use in treatment of HNSCC in a subject in need thereof.

In some embodiments, the miR-193a precursor sequence comprises SEQ ID NO: 1.

In some embodiments, the expression vector is adeno-associated expression vector (AAV).

In some embodiments, the HNSCC is hypopharynx squamous cell carcinoma.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a non-limiting schematic representation of the pAD-EF1α mammalian expression vector.

FIG. 2 includes a vertical bar graph of cell viability following miR-193a-5p plasmid transfection. Squamous cancer cells Fadu were transfected with pCMV-miR-193a, or with p-CMV-GFP (MOC plasmid), 48 hours post transfection. cell viability was detected by XTT. Presented data are means±SD of three experiments, each conducted in eight replicates, and are expressed as percentages of the respective control. ***P<0.001(Dunnett's test).

FIG. 3 includes a vertical bar graph of cell viability following miR-193a-5p plasmid transfection time course experiment. Squamous cancer cells Fadu were transfected with miR-193a-5p or with MOC plasmid, 24 hours, 48 hours and 72 hours post transfection. cell viability was detected by XTT. Presented data are means±SD of three experiments, each conducted in eight replicates, and are expressed as percentages of the respective control. Statistical significance determined by a two-tailed student's t-test. Significance was determined as follows: *P<0.05, **P<0.01 and ***P<0.001.

FIGS. 4A-4B represent cell cycle distribution of treated cells. 1×10⁶ Fadu cells were implanted into six well plates, 24 hours later cells were transfected with either CMV-GFP plasmid (control group) or CMV-miR-193a plasmid at 3 μg/well concentration for 48 hours. Cells were then harvested, fixed and stained with propidium iodide (PI) and subjected to a cell cycle analysis using FACS Calibur. The distribution and percentage of cells in the Sub-G1 phase of the cell cycle are presented. (4A) Representative histograms of three experiments. (4B) A vertical bar graph of cell in Sub-G1 of each experiment. 3-5 repeats each (cells were pooled before staining). The control group was transfected with CMV-GFP plasmid and the treatment group was transfected with CMV-miR-193a plasmid. The AVG bars are the average between the three experiments. Statistical significance was determined (***P<0.001(Dunnett's test)).

FIG. 5 includes a vertical bar graph of apoptotic cells induced by miR-193a transfection and determined by Annexin V and PI. 1×10⁶ Fadu cells were implanted into six well plates, 24 hours later cells were transfected with either CMV-GFP plasmid (control group) or CMV-miR-193a plasmid at 3 μg/well concentration for 48 hours. Cells were then harvested and stained with Annexin V-FITC and PI and analyzed by flow cytometry. Data presented are average of three independent experiments each conducted in duplicates [mean±SE]. Statistical significance determined by a two-tailed student's t-test (treatment vs. control). ***P<0.001.

FIG. 6 includes a graph presenting data from 16 athymic nude male mice which were subcutaneously injected with 1×10⁶ Fadu cells. When the tumors reached a volume of 200 mm³ the mice were divided into 2 groups of 8 mice each, with a similar dispersal of tumor volumes. Mice were injected once a week with intraperitoneal (IP) injection of either PBS×1 to the control group, or 1×10¹⁰ vp AD-miR-193a, for 3 weeks. During the experiments, tumor volumes were measured twice a week using caliber meter. The results presented are the mean±SE. n=8 mice per experimental group.

FIGS. 7A-7B include vertical bar graphs showing the effect of miR-193a on final tumor weight (7A) and volume (7B). The results presented are the mean±SE. n=8 tumors per experimental group. Statistical significance for tumor weight determined by Mann-Whitney and for tumor volume by two-tailed Student's t-test (treatment vs. control). ***P<0.01.

FIG. 8 includes a vertical bar graph showing Real-time analysis of miRNA expression patterns in Fadu Cells. The expression levels of miRNAs; hsa-mir193a were analysed using Quantitative real-time PCR, following 5-aza-2′ -deoxycytidine cell treatment. Data represented as average of three independent experiments conducted in triplicates (mean±SD) and U6 was used as normalization. Statistical significance determined by a two-tailed Student's t-test. Significance was determined as follows: ***P<0.01.

FIGS. 9A-9B include graphs showing the expression levels of miR-193a-3p in HNSCC tissues. Quantitative RT-PCR analysis of the relative expression levels of miR-193a in 7 pairs (total 14 samples) of HNSCC cancer tissues and normal adjacent tissue samples. Total RNA was extracted from formalin-fixed paraffin (FFP) blocks and the expression levels of miRNA-193a were analysed using Quantitative real-time PCR (n=7each). (9A) Circles represent miR-193a folds of expression from U6 in normal tissues, whereas squares represent miR-193a folds of expression from U6 control in patients' tissue. (9B) Averaged data presented in 9A and presented as means±SE. Statistical analysis was performed using Student's t-test with ***P<0.001 compared to control group.

### DETAILED DESCRIPTION

The present invention, in some embodiments, provides expression vectors comprising miR-193a for use in treating cancer in a subject in need. In some embodiments the invention provides compositions for treatment and methods of diagnosing and/or treating HNSCC disease using miR-193a.

According to some embodiments, the invention is based on the surprising findings that miR-193a induced apoptosis in Fadu cells.

A skilled artisan would appreciate that FaDu cells are derived specifically from a squamous cell carcinoma of the hypopharynx. FaDu cells are specifically used as an in vitro cell model of hypopharynx squamous cell carcinoma. In some embodiments, FaDu cell are not a general head and neck cancer an in vitro cell model.

Therefore, the present invention is based, in part, on the use miR-193a to specifically, primarily, predominantly, or any combination thereof, treat head and neck squamous cell carcinoma in a subject in need thereof.

As used herein, “specifically”, “primarily”, or “predominantly” is to denote a higher efficacy in treatment of HNSCC over other cancer types. In some embodiments, “specifically”, “primarily”, or “predominantly” is to denote a higher efficacy in treatment of HNSCC over other head and neck cancer types. Non-limiting examples of other head and neck cancer types include, but are not limited to, oral or mouth cancer, nose cancer or paranasal sinus and nasal cavity cancer, laryngeal cancer, trachea cancer, or others.

Further, carcinoma of the hypopharynx often have an advanced stage at diagnosis. Occasionally, carcinoma of the hypopharynx have the most adverse prognoses of pharyngeal tumors.

As a result of a vast lymphatic system at the proximity of the larynx, hypopharynx carcinomas may metastasize at very early stages.

Therefore, a biomarker for early detection, e.g., expression level of miR-193a, is of importance for early diagnosis, prognosis, treatment, or any combination thereof

To date, there is only an elemental understanding of the molecular, cellular and environmental mechanisms that drive HNSCC pathogenesis, and there are only limited therapeutic options, many with negligible clinical benefit. Therefore, the findings herein provide using miR-193a as gene therapy in head and neck cancer.

Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

According to some embodiments, pri-miRNA is further processed to miRNA by known molecular mechanisms of RNA interference. For example, the known RNA-induced silencing complex (RISC).

miRNA base pairing to a site in the target mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

According to some embodiments, the polynucleotide comprises a mir-193a precursor sequence.

According to some embodiments, miR-193a sequence is at least 70%, 80%, 90%, or 100% identical to SEQ ID NO:1. According to some embodiments, miR-193a-5p sequence is at most 70%, 80%, 90%, or 99% identical to SEQ ID NO: 1. Each possibility represents a separate embodiment of the invention.

SEQ ID NO:1: Homo sapiens miR-193a: UGGGUCUUUGCGGGCGAGAUGA.

Expression Vector

In some embodiments, there is provided an expression vector comprising a promoter operably linked to a polynucleotide having a miR-193a precursor sequence.

In some embodiments, the expression vector is for use in the treatment of cancer in a subject in need thereof.

In some embodiments, the promoter is an inducible or constitutive promoter.

In some embodiments, the promoter is a tissue specific promoter.

In some embodiments, the promoter is a cell specific promoter.

In some embodiments, the promoter is activated, e.g., drives expression of a gene or a polynucleotide operably linked thereto, in a cancerous cell. In some embodiments, the cancerous cell is a squamous cell. In some embodiments, the squamous cell is a cell of the hypopharynx.

Non-limiting examples of promoters activated in a squamous cell of the hypopharynx or a cancerous cell of the hypopharynx include, but are not limited to, the promoter of the genes: eukaryotic Translation Initiation Factor 4 Gamma 1 (EIF4G1), dishevelled segment polarity protein 3 (DVL3), Ephrin type-B receptor 4 (EPHB4), Minichromosome Maintenance Complex Component 7 (MCM7), Breast cancer metastasis suppressor 1 (BRMS1), Spliceosome Associated Factor 1 (SART1), to name a few.

In some embodiments, a promoter operably linked to SEQ ID NO: 1, e.g., driving the expression of the latter in a hypopharynx squamous cell carcinoma, is selected from: EIF4G1, DVL3, EPHB4, MCM7, BRMS1, or SART1.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid” which refers to a circular double stranded DNA loop into which additional DNA segments can be inserted, such as by standard molecular cloning techniques. Another type of vector, wherein virally-derived DNA or RNA sequences are present in the virus (e.g. retroviruses, replication defective retroviruses, adenoviruses, replication defective adenoviruses, and adeno-associated viruses). Viral vectors also include polynucleotides carried by a virus for transfecting into host cells. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked.

Such vectors are referred to herein as “expression vectors”. Common expression vectors of utility in recombinant DNA techniques are often in the form of plasmids.

The term “expression” as used herein refers to the biosynthesis of a gene product, including the transcription of the gene product. Thus, expression of a nucleic acid molecule may refer to transcription of the nucleic acid fragment (e.g., transcription resulting in mRNA or other functional RNA).

Recombinant expression vectors include one or more regulatory elements, which may be selected on the basis of the host cells to be used for expression, that is operatively-linked to the nucleic acid sequence to be expressed. Within a recombinant expression vector “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

In one embodiment, the present invention provides a vector or a plasmid comprising the nucleic acid molecule as described herein. In one embodiment, a vector or a plasmid is a composite vector or plasmid. In one embodiment, a vector or a plasmid is a man-made vector or plasmid comprising at least one DNA sequence which is artificial. In one embodiment, the present invention provides a vector or a plasmid comparing: Adeno Associated Virus, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In one embodiment, the present invention provides a vector or a plasmid comprising regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

According to some embodiments, a recombinant adeno-associated vector (AAV) comprising one or more polynucleotide sequence encoding the VEGF, VEGF-stimulating compound, VEGFR-stimulating compound, or any combination thereof, is provided.

In one embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In one embodiment, it will be appreciated that the polypeptides of the present invention can also be expressed from a nucleic acid construct administered to the individual employing any suitable mode of administration, described hereinabove (i.e., in-vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

Pharmaceutical Composition

According to one aspect of the invention, there is provided a pharmaceutical composition comprising a miR-193a precursor and a pharmaceutically acceptable carrier, for use in the treatment of HNSCC.

According to one aspect of the invention, there is provided a pharmaceutical composition comprising a miR-193a precursor and a pharmaceutically acceptable carrier.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman' s: The Pharmacological Bases of Therapeutics, 8th Ed., Gilman et al. Eds. Pergamon Press (1990); Remington's Pharmaceutical Sciences, 18th Ed., Mack Publishing Co., Easton, Pa.

(1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

According to some embodiments, the pharmaceutical composition is used in treating cancer in a subject in need thereof.

Methods of Treatment

In some embodiments, there is provided a method of treating or ameliorating a subject afflicted with head and neck squamous cell carcinoma, the method comprising administering to the subject a composition comprising a therapeutically effective amount of a miR-193a precursor.

In some embodiments, there is provided a method for treating or ameliorating a subject afflicted with HNSCC, the method comprising administering to the subject a composition comprising a therapeutically effective amount of miR-193a or a precursor thereof, thereby treating or ameliorating the subject afflicted with HNSCC.

According to some embodiments, there is provided a method for diagnosing and/or prognosing HNSCC in a subject, the method comprising determining the expression levels of a miR-193a in a sample obtained or derived from the subject, wherein a significant difference in the expression levels of miR-193a compared to a control is indicative of a diagnosis or prognosis of HNSCC in said subject.

In some embodiments, the expression level of miR-193a is downregulated or reduced compared to the control.

According to some embodiments, cell viability is decreased by at least 10%, 20%, 30%, 40%, 50%, or 60%. According to some embodiments, cell viability is decreased by at most 99%, 90%, 80%, 70%, 60%, 50% or 40%. According to some embodiments, cell viability is decreased by 20-80%, 30-70%, 40-60%, 50-60%, 10-40%, or 15-40%. Each possibility represents a separate embodiment of the invention.

As used herein “cancer” or “pre-malignancy” are diseases associated with cell proliferation. Non-limiting types of cancer include carcinoma, sarcoma, lymphoma, leukemia, blastoma and germ cells tumors. In one embodiment, carcinoma refers to tumors derived from epithelial cells including but not limited to breast cancer, prostate cancer, lung cancer, pancreas cancer, and colon cancer. In one embodiment, sarcoma refers of tumors derived from mesenchymal cells including but not limited to sarcoma botryoides, chondrosarcoma, Ewing's sarcoma, malignant hemangioendothelioma, malignant schwannoma, osteosarcoma and soft tissue sarcomas. In one embodiment, lymphoma refers to tumors derived from hematopoietic cells that leave the bone marrow and tend to mature in the lymph nodes including but not limited to Hodgkin lymphoma, non-Hodgkin lymphoma, multiple myeloma and immunoproliferative diseases. In one embodiment, leukemia refers to tumors derived from hematopoietic cells that leave the bone marrow and tend to mature in the blood including but not limited to acute lymphoblastic leukemia, chronic lymphocytic leukemia, acute myelogenous leukemia, chronic myelogenous leukemia, hairy cell leukemia, T-cell prolymphocytic leukemia, large granular lymphocytic leukemia and adult T-cell leukemia. In one embodiment, blastoma refers to tumors derived from immature precursor cells or embryonic tissue including but not limited to hepatoblastoma, medulloblastoma, nephroblastoma, neuroblastoma, pancreatoblastoma, pleuropulmonary blastoma, retinoblastoma and glioblastoma-multiforme. In one embodiment, germ cell tumors refers to tumors derived from germ cells including but not limited to germinomatous or seminomatous germ cell tumors (GGCT, SGCT) and nongerminomatous or nonseminomatous germ cell tumors (NGGCT, NSGCT). In one embodiment, germinomatous or seminomatous tumors include but not limited to germinoma, dysgerminoma and seminoma. In one embodiment, nongerminomatous or nonseminomatous tumors refers to pure and mixed germ cells tumors including but not limited to embryonal carcinoma, endodermal sinus tumor, choriocarcinoma, tearoom, polyembryoma, gonadoblastoma and teratocarcinoma.

In some embodiments, there is provided a method of treating or ameliorating a subject afflicted with cancer, the method comprising administering to the subject a composition comprising a therapeutically effective amount of a miR-193a precursor.

According to some embodiments, the cancer disease is selected from the group consisting of leukemia, hepatocarcinoma, lung epithelial carcinoma, cervical adenocarcinoma, and Squamous cancer.

According to some embodiments, the cancer disease is Head and neck squamous cell carcinoma (HNSCC).

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the terms “administering,” “administration,” and like terms refer to any method which, in sound medical practice, delivers a composition containing an active agent to a subject in such a manner as to provide a therapeutic effect. One aspect of the present subject matter provides for dermal or transdermal administration of a therapeutically effective amount of a composition of the present subject matter to a patient in need thereof. Other suitable routes of administration can include oral, dermal, transdermal, parenteral, subcutaneous, intravenous, intramuscular, or intraperitoneal. In some embodiments, the administering is systemic administering. In some embodiments, the administering to the wound. In some embodiments, the administering is to the site of inflammation.

Administering the composition to a specific site in the subject may be performed with any method known in the art. This may include with an applicator, in the form of a gel or cream, as well as on a scaffold, wrap or bandage.

The dosage administered will be dependent upon the age, health, and weight of the recipient, kind of concurrent treatment, if any, frequency of treatment, and the nature of the effect desired.

The term “significant difference” in the context of the measured expression levels includes up-regulation and/or down-regulation, or combinations thereof of examined genes.

In some embodiments, the significant difference is a statistically significant difference such as in mean expression levels, as recognized by a skilled artisan. For example, without limitation, an increase or a decrease of about at least two folds, or alternatively of about at least three folds, compared to a control value is associated with a specific cancer.

According to some embodiments, miR-193a is downregulated compared to the control.

The term “comparable” or “corresponding” in the context of comparing the expression levels of genes to a control sample means that the same type of sample is used in the comparison. For example, expression level of miR-193a from a HNSCC sample can be compared to an expression level of miR-193a in another sample, such as a neighboring/adjacent sample. In another embodiment, the control sample may be obtained from the same subject. In another embodiment, the control sample is obtained from adjacent tissues (i.e., tissue adjacent to the tumor). In some embodiments, comparable samples may be obtained from the same individual at different times, such as for monitoring the efficacy of various therapies and/or preventive interventions. In other embodiments, comparable control samples may be obtained from different individuals (e.g., a patient and a healthy individual).

In general, samples may be normalized by a common factor. For example, cell-containing samples are normalized by protein content or cell count. In some embodiments, samples are normalized using a set of normalization genes.

The term “normalized” with regard to a gene transcript or a gene expression product refers to the level of the transcript or gene expression product relative to the mean levels of transcripts/products of a set of reference genes, wherein the reference genes are either selected based on their minimal variation across, patients, tissues or treatments (“housekeeping genes”), or the reference genes are the totality of tested genes.

In another embodiment, the method further comprises normalizing the expression levels of the miR-193a against a level of at least one reference RNA transcript in the tissue sample to provide a normalized expression level of the miR-193a. In one embodiment, a significant difference of the normalized expression level of miR-193a compared to a control is an indication of a diagnosis or prognosis of cancer in a subject.

As used herein, the term “reference level” refers to a level of a substance which may be of interest for comparative purposes. In one embodiment, a reference level may be the expression level of a nucleic acid expressed as an average of the level of the expression level of a nucleic acid from samples taken from a control population of healthy (disease-free) subjects. In another embodiment, the reference level may be the level in the same subject at a different time, e.g., before the present assay, such as the level determined prior to the subject developing the disease or prior to initiating therapy.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

Gene expression is the transcription of DNA into messenger RNA by RNA polymerase. Up-regulation describes a gene which has been observed to have higher expression (higher RNA levels) in one sample (for example, from cancer tissue) compared to another (usually healthy tissue from a control sample). Down-regulation describes a gene which has been observed to have lower expression (lower RNA levels) in one sample (for example, from cancer tissue) compared to another (usually healthy tissue from a control sample).

Numerous other methods are known in the art for measuring expression levels of a one or more gene such as by amplification of nucleic acids (e.g., PCR, isothermal methods, rolling circle methods, etc.). The skilled artisan will understand that these methods may be used alone or combined. A common technology used for measuring RNA abundance is RT-qPCR where reverse transcription (RT) is followed by real-time quantitative PCR (qPCR). Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues (Nolan T, et al. Nat Protoc 1:1559-1582, 2006; Paik S. The Oncologist 12:631-635, 2007; Costa C, et al. Transl Lung Cancer Research 2:87-91, 2013).

As used herein, the term “about” when combined with a value refers to plus and minus 10% of the reference value. For example, a length of about 1000 nanometers (nm) refers to a length of 1000 nm+- 100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cell—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods

Subcloning of miR-193 Nucleotide Sequence into a Mammalian Expression Cassette Containing the EF1a Promoter.

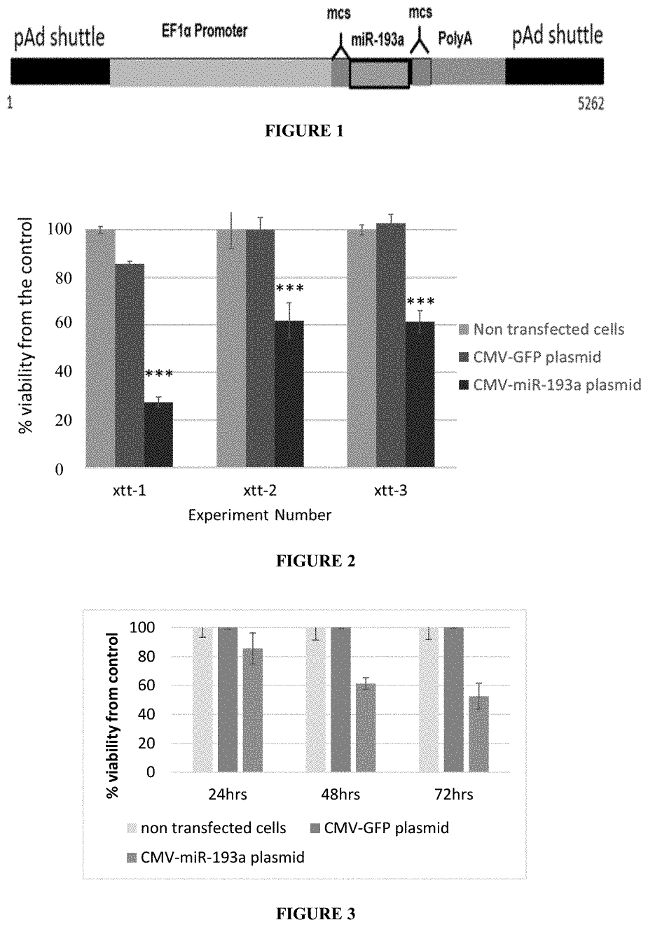

The miR-193a precursor sequence was prepared and sequenced by OriGene Technologies Inc. The pre-miR 193a was amplificated by PCR and subcloned into pAd shuttle vector (4.6 kb; FIG. 1).

Transformation of the CMV-miR-193a Plasmid into JM 109 E. Coli Competent Cells

For transformation, 1 μl of 100 ng/μl pCMV-miR-193 were mixed with 100 μl JM109 competent cells, the transformation was done according to the heat shock protocol (Promega), the transformed solution at the end was plated on LB agar plates containing 100 μg/ml Kanamycin. 10 μl, 25 μl, 50 μl or 100 μl of the transformation products were plated on LB plates containing 100 μg/ml Kanamycin and incubated overnight in 37° C. incubator.

CMV-miR-193a Plasmid Extraction and Verification

2 colonies were picked and transferred into fresh LB 100 μg/ml Kan plate (colonies from the 10 μl plate) colonies were also inoculated into two 50 ml conical tubes containing 5 ml LB media supplemented with 100 μg/ml Kan. For incubation, the tubes were placed in 37° C. incubator with shaking at 220 rpm. Plasmids were extracted using the Promega “Wizard Plus Miniprep DNA Purification System” kit according to the manufacturer protocol. In last step, plasmids were eluted from the column with 50 μl DEPC water (at 64° C.).

Amplification of the pre-miR-193a by PCR

In order to sub-clone the pre miR-193 sequence into the pAD-EF1a shuttle vector, primers contain Hindlll recognized sequence in the 5′-prime and the BamHI recognized sequence in the 3′-prime were synthesized. The CMV-miR-193a plasmid was used as the PCR template, for negative control no template was used.

Primers sequence:

| | |
|---|---|
| Forward primer with HindIII | CGAAGCTTCTGCGGTGGGGAGG |
| recognized sequence (AAGCTT) | CTTTGGT (SEQ ID NO: 2) |
| Reverse primer with BamHI | CCGGATCCGGTCCCGTCTGTCCA |
| recognized sequence (GGATCC) | CTCAACC (SEQ ID NO: 3) |

Digestion of pre-miR-193a (Amplificated by PCR) and pAd-Ef1a Shuttle Vector with BamHI and HindIII Restriction Enzymes

The insert was purified after PCR using Promega PCR purification kit according to the manufacturer procedure and then digested with BamHI and Hindlll restriction enzymes. pAD-EF1a shuttle vector also was digested with similar enzymes as the PCR insert.

Extraction of the Cleaved Plasmid and the Insert from 0.7% Agarose Gel

The digested DNA samples were loaded onto a 0.7% agarose gel, separated, and the pre-miR-193a 642 bp DNA fragment and the pAD-EF1a 5.2 kb DNA fragment were excised from the gel and extracted from the agarose. Using a gel extraction kit (QIAquik gel extraction kit from QIAGENE), the purified plasmid and insert where loaded into electrophoresis 1% agarose gel after serial dilutions and the concentration was also measured using nanodrop before ligation.

Ligation and Transformation into JM109 Competent Cells

Once isolated the purified vector (pAd-Ef1a) and insert (pre-miR-193a) were ligated together using T4 DNA Ligase (Roche) with ratio 1:3 between vector and insert. 7 μl of the ligation were transformed into JM109 competent cells, a day later more than 30 colonies were grown in the ligation plate, weather no colonies were grown in the negative control plate. Eight colonies were isolated a day later.

Colonies Screening for Cloned Insert

plasmid DNA was isolated from eight colonies and digested with HindIII and BamHI to verify the pre-miR-193a insert presence in the plasmid.

Conformation of Cloning by Sequencing

To confirm that the pre-mir-193A was subcloned correctly, DNA from colonies 1 and 2 were sequenced. Both constructs contained the pre miR-193a sequence and confirmed that no unwanted changes were introduced.

Vector Amplification

One 30×145 mm dishes of HEK 293 cells of 80% confluent, were infected with starter of Ad-miR-193a (provided by Abm, AdmiRa-hsa-mir-193a Virus cat#: mh0241). Two days later, the dishes infected with Ad-CMV-PDX1 showed complete cytopathogenic effect. Cells and medium with cells were harvested, followed by three cycles of freeze and thaw. 30×145 mm dishes of 293 cells of 80% confluent, were infected with 0.5 ml of crude virus from first amplification. 48 hours later all the infected dishes showed complete CPE. Cells were scrapped with media and transferred into 50 ml vials, cells were than pelleted by 3,000 g centrifugation and stored freeze at −80° C. for vector purification.

Virus Purification

Infected cell pellet was lysed using 5% sodium deoxycholate followed by DNase I and RNase I treatment. Both fractions were loaded separately on two-step CsCl gradient in SW40 tubes. After 3 hours of centrifugation, the viral bands (lower band) were collected and loaded on continuous CsCl gradient in SW55 tubes and centrifuged overnight. The viral bands were retrieved and dialyzed overnight at 4° C. with 3×500 ml changes of 10 mM Tris-HCL pH 8.0, 10% glycerol was added to the adenovirus post dialysis. Virus was aliquoted in 47 cryogenic vials (100 μl/vial) and frozen at −80° C.

miRNA Isolation and Quantification and Profiling

miRNA was isolated using mirVana™ miRNA Isolation Kit (Ambion®) according the manufacturer's protocol, the mirVana™ kit utilizes two sequential GFFs, since the small

RNAs are essentially lost in the first filtration through the column and therefore a second filtration is required to capture the tiny microRNAs. miRNA was eluted in 50 μl RNase-free water.

The concentration and purity of miRNA was assessed using NanoDrop™ lite spectrophotometer (NanodropTechnologies, Willmington, Del., USA). miRNA expression profiling was performed using the TaqMan microRNA arrays system (Applied Biosystems) with specific TaqMan primers and probes for miR-193a. cDNA was generated using TaqMan microRNA assay using Primers for the mir-193a with FAM/MGB. U6 was used as internal expression control. Real time PCR was carried out with Rotor Gene 6000 Real-Time PCR Machine-Bosch-Bosch, results were analyzed with delta delta Ct (ΔΔCt) method using the rotor Gene 6000 Real-Time PCR software.

### EXAMPLE 1

### Viability Assay Following Transient Transfection of Fadu Cells with the CMV-miR-193a Plasmid

Fadu Cells were seeded in a 96 well plate (3×10⁴ cells/well). Twenty-four hours later, cells were transfected with 1 μg of pCMV-miR-193a plasmid and 2.5 μl well Lipofectamine 2000 and incubated for 48 hours (for negative control cells were transfected with 1 μg p-CMV-GFP plasmid)

At the end of treatment, cell viability was measured using XTT. The data presented are average of at least three independent experiments; 8 repeats each (means±SEM), and are expressed as percentage of the respective vehicle treated control. As seen in FIG. 2, Fadu cells were highly sensitive to miR-193a transfection. Cell viability was reduced to approximately 70% in the first experiment and to 40% in the second and third experiment. No effect of viability was demonstrated in the cells transfected with MOC plasmid.

### EXAMPLE 2

Viability Time Course Experiment Following Transient Transfection of Fadu Cells with the CMV-miR-193a Plasmid

Transient transfection has a maximum expression of the plasmid at between 48-72 hours therefore transfection experiments were repeated, and cell viability was tested in three different time points (24 h, 48 h and 72 h). Fadu Cells were seeded in a 96 well plate (3×10⁴ cells/well). Twenty-four hours later, cells were transfected with 1 μg of pCMV-miR-193a plasmid and 2.5 μl well Lipofectamine 2000 and incubated for 24, 48 and 72 hours (for negative control cells were transfected with 1 μg p-CMV-GFP plasmid). At the end of treatment, cell viability was measured using XTT. Fadu cell's viability was decreased in approximately 15%, 40%, and 50% following transfection with CMV-miR-193a plasmid after a period of 24, 48 and 72 hours, respectively (FIG. 3).

### EXAMPLE 3

Cell Cycle Analysis

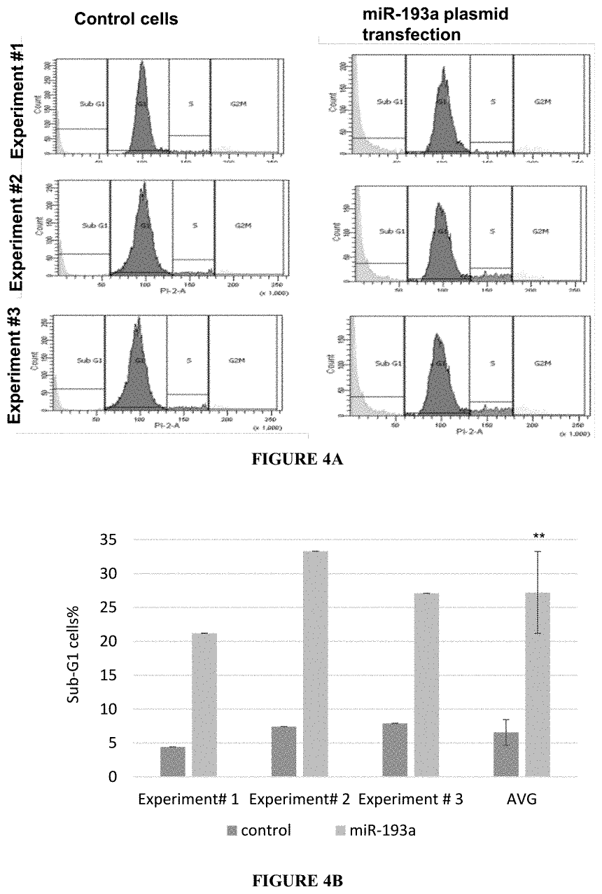

To further characterize the effect of the transfection with CMV-miR-193 on Fadu cells, cell cycle analysis was conducted for the evaluation of cell death. Apoptotic cells often have fractional DNA content due to DNA degradation by the apoptosis-associated nucleases. Additionally, some apoptotic cells also lose DNA (chromatin) by shedding apoptotic bodies. Therefore, apoptotic cells were identified within a population as the cells that evidence fractional DNA content following extraction of the degraded DNA and subsequent cell staining with PI. Apoptotic cells were then represented on the DNA content frequency histograms by the “sub-G1” peak (FIG. 4A). In all the transfection experiments examined a significant increase in the sub-G1 phase of the cell cycle was observed 48 hours post transfection. Also, cell cycle arrest was observed in the first experiments having 5 folds increase in percent of the cells in G2M.

### EXAMPLE 4

### Quantification of Apoptotic Cells by Annexin V-FITC

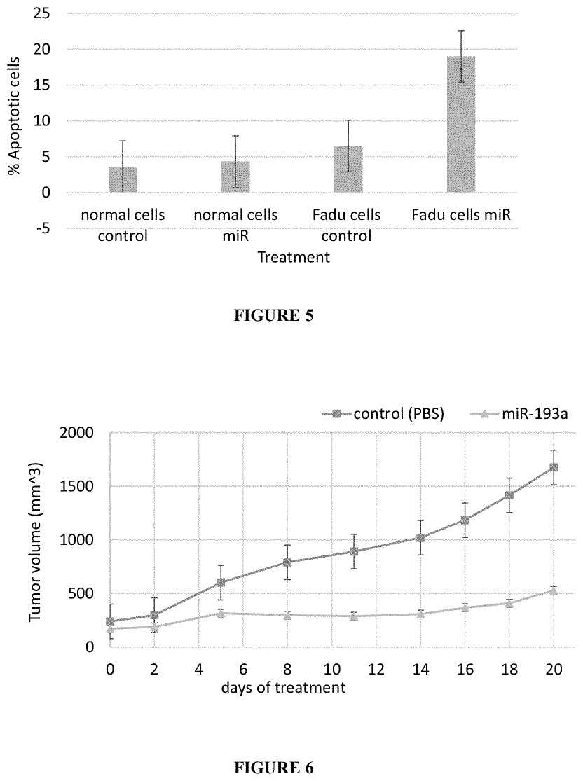

In the early stages of apoptosis, plasma membrane alterations occur at the cell surface and phosphatidylserine (PS) translocates from the inner side of the plasma membrane to the outer layer. Surface PS is detectable by flow cytometry using Annexin V-FITC, a Ca²⁺-depended phospholipid binding protein with high affinity to PS. Annexin V-FITC and PI staining were used in order to quantify the percentage of viable cells and cells undergoing apoptosis or necrosis. Fadu cells and normal fibroblasts cells were transfected with miR-193a plasmid or GFP plasmid as a control, 24 hours later Annexin V and PI staining were done according to the manufacturer report. The results revealed that miR-193a induced apoptosis, on Fadu cell line, compared with control cells. According to FIG. 5, the number of apoptotic cells in Fadu increased by 3 folds after 24 hours treatment, when compared to control cells. Significant increase in apoptosis rate was detected (P<0.001).

### EXAMPLE 5

### The Effect of Overexpression of miR-193a-5p on HNSCC Tumor Cells Growth in Mice

Xenograft animal model were generated via injection of 1×10⁶ Fadu cells in Matrigel subcutaneous in athymic nude mice immunodeficient mice. When the tumors reached a volume of 200 mm³ the mice were divided into 2 groups of 8 mice each, with a similar dispersal of tumor volumes. 7 and 14 days post implantation, mice were injected with either PBS×1 to the control group, or 1×10¹⁰ vp AD-miR-193a.

Tumors were measured by electronic calliper, once a week. 21 days post treatment the tumors were harvested and weight was measured. Control mice tumor size was significantly larger compared to the tumor size of mice treated with AD-miR-193a (FIG. 6).

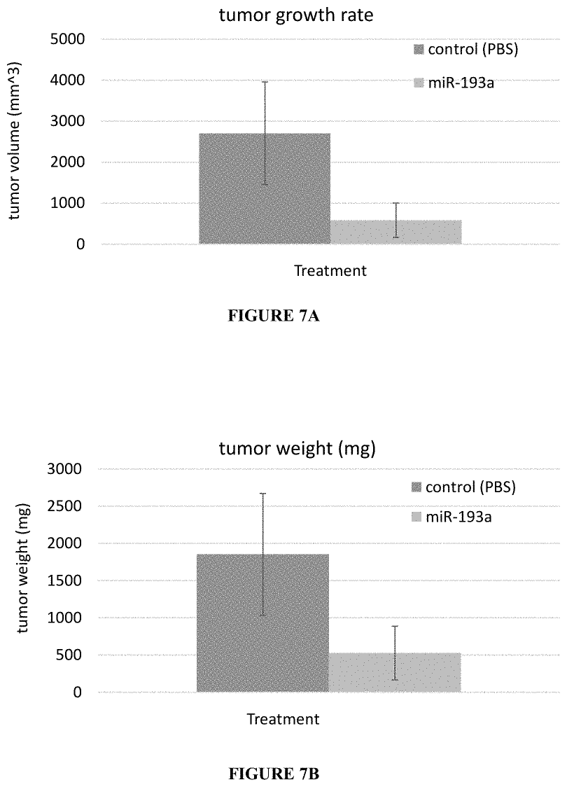

The animals were sacrificed, and the tumors were removed, weighed and measured. FIG. 7 shows that there were statistically significant differences between the final tumor weights (FIG. 7A) and volumes (FIG. 7B). Results show that treatment with miR-193a significantly reduced the weight and the tumor volume in mice when compared to the control group. (P<0.0001).

### EXAMPLE 6

### miR-193a Expression Levels in Fadu Cells Following AD-miR-193a

To test the expression levels of miR-193a in Fadu cells, Squamous cancer cells Fadu were treated after 24 hours in culture with increasing 5 μM of 5-aza-2′ -deoxycytidine for a period of 48 hours, squamous cancer cells Fadu were treated after 24 hours in culture with 5 μM of 5-aza-2′ -deoxycytidine, or were transduced with 1E9 vp of AD-miR-193 for a period of 48 hours; control cells were treated with 0.01% DMSO.

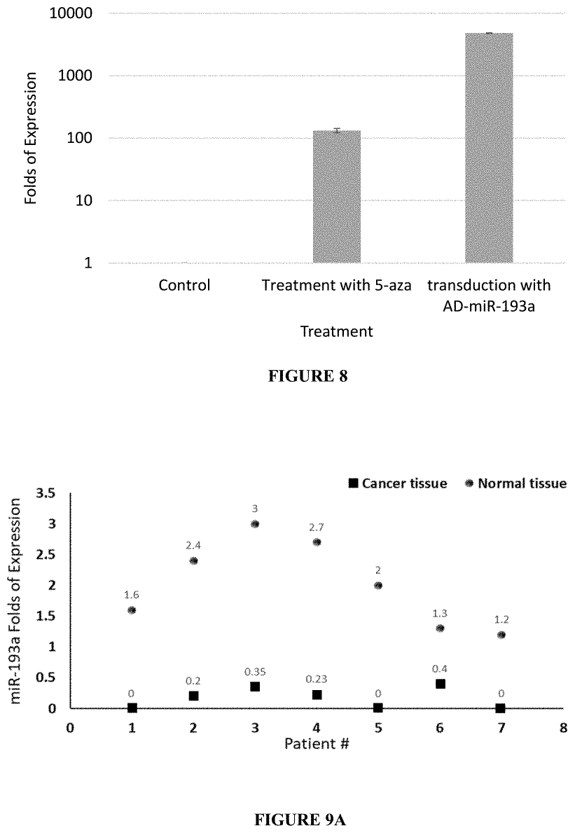

miRNA were isolated, quantified, profiled and cDNA was generated. The results showed that Fadu control cells did not express the miR-193a. Treatment with 5-aza increased the expression levels by 132-folds, while transduction with AD-miR-193a increased the expression levels by 4,817-folds (FIG. 8).

### EXAMPLE 7

### Personalized Treatment

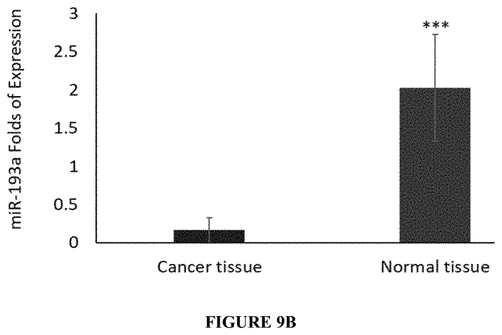

In order to assess the expression patterns of miR-193a in patients' tissues, 7 HNSCC tissues and 7 normal adjacent tissues were collected from FFTP blocks. miR-193a levels were determined using quantitative RT-PCR miR-193a levels and the U6 micro RNA as internal control. The results show that miR-193a was consistently downregulated in cancer tissues compared to normal adjacent tissues (FIG. 9A). Three out of seven of the cancer tissues did not express miR-193 at all, and the expression levels were zero.

When looking at the average of miR-19a expression, it was found that in normal tissue, miR-193/U6 control ratio level was about 2.02+0.69. However, in patients' tissues, the relative miR-193a expression average was 0.16+0.16 (FIG. 9B).

It is concluded that a downregulated expression level of miR-193a is indicative of HNSCC. Accordingly, it is suggested and exemplified herein, that elevating miR-193a levels in hypopharynx squamous cell carcinoma in vivo, provides therapeutic effects for HNSCC pathology.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1.-6. (Cancelled)

7. A method of treating or ameliorating a subject afflicted with head and neck squamous cell carcinoma (HNSCC), the method comprising the steps of:
a. determining the level of micro RNA (miR)-193a in a sample obtained or derived from said subject, wherein downregulated expression of said miR-193a compared to a control is indicative of the subject is suitable for treatment using miR-193a; and
b. administering a therapeutically effective amount of said miR-193a to said subject, thereby treating or ameliorating a subject afflicted with HNSCC.

8. The method of claim 7, wherein said HNSCC is hypopharynx squamous cell carcinoma.

9. The method of claim 7, wherein said miR-193a is administered as an expression vector comprising a promoter operably linked to a polynucleotide having a miR-193a precursor sequence.

10. The method of claim 9, wherein said miR-193a precursor sequence of said expression vector comprises SEQ ID NO: 1.

11. The method of claim 9, wherein said expression vector is an adeno-associated expression vector (AAV).

12. A pharmaceutical composition comprising an effective amount of any one of:
a. miR-193a or a precursor thereof; and
b. the expression vector administered according to the method of claim 9, and a pharmaceutically acceptable carrier.
